(19) United States
(12) Patent Application Publication
(10) Pub. No.: US 2013/0138183 A1
(43) Pub. Date: May 30, 2013

(54) Methods of Transferring Energies to Water, Alcohols and Minerals

(71) Applicant: William John Martin, South Pasadena, CA (US)

(72) Inventor: William John Martin, South Pasadena, CA (US)

(21) Appl. No.: 13/304,558

(22) Filed: Nov. 25, 2011

(51) Int. Cl. A61N 5/06; C12G 3/08; G21K 5/00; A61K 33/08

(52) U.S. Cl. 607/94; 424/692; 426/592; 426/248; 250/492.1

(57) ABSTRACT

The use of humic acids, zeolites, magnesium oxide beads and other mineral-containing materials in the activation of ethanol, alcoholic beverages and water is disclosed. Consumption of the energized liquids can have therapeutic benefits. The activated ethanol can be further used with neutral red dye and ultraviolet (UV) light illumination to indirectly enhance the environmental energy absorption properties of other liquids, including drinking water and of mineral containing materials. Mineral activated ethanol can similarly be used with neutral red dye and UV illumination to enhance the alternative cellular energy (ACE) pathway of an individual.

### CROSS REFERENCE TO RELATED APPLICATIONS

### Co-Pending and Previously Submitted Patent Application

Methods for detection of ultraviolet light reactive alternative cellular energy pigments (ACE-pigments). William John Martin Submitted Dec. 24, 2007. Publication number 20090163831

Method of assessing and of activating the alternative cellular energy (ACE) pathway in the Therapy of Diseases. William John Martin Submitted Jan. 16, 2008. Publication number 20090181467

Enerceutical mediated activation of the alternative cellular energy (ACE) pathway in the therapy of diseases. Submitted May 8, 2008. Publication number 20090280193 Regenerative wound healing using copper-silver citrate composition. Publication number: 20100099758 Submitted Oct. 22, 2008.

Enerceutical activation of the alternative cellular energy (ACE) pathway in therapy of diseases. Submitted Feb. 11, 2009. Publication number 20090202442

Method of using the body's alternative cellular energy pigments (ACE-pigments) in the therapy of diseases Submitted Feb. 20, 2009. Publication number 20100215763 Urine as a source of alternative cellular energy pigments (ACE-pigments) in the assessment and therapy of diseases. Submitted Mar. 5, 2009. Publication number 20100196297

Moring a oil mediated activation of the alternative cellular energy pathway in the therapy of diseases. Submitted Feb. 24, 2010. Publication number 20110208110 Diagnostic value of systemic ACE pathway activation in the detection by fluorescence of localized pathological lesions. Submitted Jul. 26, 2010. Publication number 20100291000

Enerceutical mediated activation of the alternative cellular energy (ACE) pathway in the therapy of diseases. Submitted July 2010.

Energy Charged Liquids to Enhance Enerceutical Activation of the Alternative Cellular Energy (ACE) Pathway in the Therapy of Diseases. Submitted Dec. 17, 2010. Application Ser. No. 12/972,344

Energy Charged Alcoholic Beverages for Enhancing the Alternative Cellular Energy Pathway in the Prevention and Therapy of Diseases. Submitted January 2011.

Methods for Detecting and Monitoring the Activity of Energized Water and Other Liquids Useful for Enhancing the Alternative Cellular Energy Pathway in the Prevention and Therapy of Diseases. Submitted February 2011

Methods for Increasing the Kinetic Activity of Alcohol, Water and Other Liquids, so as to Render the Liquids More Useful in Enhancing the Alternative Cellular Energy Pathway in the Prevention and Therapy of Diseases. Submitted February 2011

Methods for Increasing the Kinetic Activity of Water and Other Liquids, so as to Render the Liquids More Useful in Enhancing the Alternative Cellular Energy Pathway and in Various Other Agricultural and Industrial Applications. Submitted June 2011. Use of Plants and Plant Extracts to Activate Water, Alcohol and Other Liquids. Submitted November 2011

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

It has been proposed that certain subtle properties of water can be modified by various methods, which typically utilize an external source of physical energy, such as magnets, electricity, vortex, sound and crystals. The reported beneficial properties of the treated and presumably “energized” water include amelioration of many human and animal illnesses; improved growth rate of plants; and reversal of corrosion of metal water pipes. How these effects are achieved is still controversial with a common oversimplification that the processed water is “restructured” into clusters, which differ in their sizes or in other qualities when compared to the clusters in unprocessed water. (It is usually assumed that the cluster size in the activated water is somewhat reduced or that the individual water molecules within the clusters are less tightly bound and even slightly expanded).

In the co-pending patent applications, incorporated herein by reference, I have disclosed the following major findings: 1. Neutral red particles sprinkled onto activated water will rapidly dissolve as linear streaks. Remaining un-dissolved material will also move throughout the solution, often in a to-and-fro manner. These movements are readily seen microscopically. Water obtained from various sources will differ in their levels of activity, with some water samples showing essentially no linear streaking of the dissolving neutral red dye, but rather slowly enlarging concentric circles of dye with stationary un-dissolved materials. The dissolving and movement patterns of neutral red (NR) dye in liquids is referred to as the NR-Kinetic assay. 2. Ethanol (absolute alcohol) and other alcohols, including alcoholic beverages, will show positive NR-Kinetic assays, when compared to water. 3. Various methods, including vortexing, stirring with a rotating magnet, exposure to sound, and bubbling through the liquid of gasses produced by electrolysis of water, will significantly enhance the NR-Kinetic activity of water, ethanol, other alcohols and alcoholic beverages. 4. Activated ethanol with neutral red dye will also fluoresce far more brightly under ultraviolet light illumination, than will regular ethanol. 5. Activated ethanol p[us neutral red dye, can lead to the activation of water and other liquids, even in the absence of direct physical contact. In one approach, the activated ethanol plus neutral red dye is placed into a sealable plastic bag, which is laid onto another liquid and the bag illuminated with a 13 Watt UV light (Halco, emitting at 365 nm, UV-A, wavelength). 6. Drinking the indirectly activated water or other beverages enhanced the sense of alertness, concentration and quality of sleep, with more vivid dreaming, of several test subjects. 7. Activated alcohol plus neutral red dye can also lead to the activation of an intrinsic alternative cellular energy (ACE) pathway used by the body in its defenses against virus infections and in wound healing. In one embodiment, a sealed plastic bag containing the activated alcohol plus neutral red dye is placed over a skin lesion, for example an outbreak of herpes simplex virus (HSV) infection and illuminated with a UV light. After several minutes, the underlying HSV skin lesion will become itself fluorescent when directly illuminated with the UV light. The direct UV illumination of the HSV skin lesion is continued till the fluorescence largely fades away (usually within 30-45 minutes. The fluorescing material within and in the vicinity of the HSV skin lesions is considered a source of cellular energy useful for the suppression of virus activity and repair of the infected cells. Another embodiment of this basic procedure is used for treating non-localized infections and especially for stealth adapted virus infections, which are not effectively recognized by the cellular immune system. In this embodiment, the plastic bag containing the activated alcohol and neutral red dye is placed onto an area of the body, such as the sole of the feet or palm of a hand and illuminated with UV light for up to an hour. Systemic activation of the ACE pathway can be confirmed by the induction or enhancement of direct UV fluorescence of the oral mucosa (tongue, palate and/or back of the throat) occurring during the treatment and persisting for some time after the treatment. Areas of induced skin fluorescence can also be occasionally observed using this procedure. Furthermore, with adults, the plastic bag containing the alcohol and neutral red dye can be placed into the mouth and directly illuminated).

In the most recent co-pending patent application, it was shown that activation of ethanol was also achievable using various plant products, including leaf powder and freshly harvested leaves and leaf powder from moring a oleifera trees. Leaves from the ashitaba plant (Angelica keiskei) could also be used. The present series of experiments were designed to address the question of whether various mineral products could also be used to directly activate water, ethanol and alcoholic beverages and, more importantly, can the level of activating activity of these mineral products be enhanced by prior exposure to UV illuminated activated ethanol with neutral red dye. The mineral containing compounds tested included humic acids, zeolites, magnesium oxide beads, touramaline and mica. Testing included the NR-Kinetic assay and clinical studies. Other incidental observations and applications of the underlying discoveries are also recorded.

### BRIEF SUMMARY OF THE INVENTION

Various mineral products were shown to increase the NR-Kinetic activity of ethanol and other fluids. Direct contact of the ethanol and the mineral product was not essential for an effect to be observed. The activating capacity of the minerals, especially humic acids, was clearly enhanced by prior placement beneath a UV illuminated plastic bag containing activated ethanol plus neutral red dye. Unprocessed and post activated mineral products have been used not only to activate drinkable water and alcoholic beverages but also E85 gasoline (containing 85% ethanol). By using a combination of methods to activate ethanol, additional aspects of the interaction of ethanol with neutral red dye have been observed, which should facilitate an understanding of the forms of energies involved in the observed phenomena.

Of particular interest was the unanticipated demonstrated activation of humic acids, zeolites and magnesium oxide beads by passage of Water Gas generated by electrolysis of water. This method was previously used to directly activate ethanol and has been used by others to treat water. The Water Gas exposed mineral containing materials were added to water and to ethanol. When compared with similar amounts of the same mineral containing materials, the Water Gas exposed material was able to achieve a greater dynamic interaction of both water and alcohol, as assessed in the NR-Kinetic assay system.

Fluid activation methods, monitored using the NR-Kinetic assay, have been extended to several additional alcoholic beverages. These have included high alcohol content spirits, including 160 proof rum (Stroh), Everclear grain alcohol (151 proof) and E85 ethanol containing gasoline. Additional activation methods have included exposure to ozone and placement of insoluble beads of magnesium oxide and touramaline and flakes of mica. Additional aspects of the interaction of neutral red dye with highly activated alcohol containing fluids have been observed. Most importantly, the newer methods have been used clinically with encouraging results. Basically, I have discovered a range of methods, used singly or in various combinations to help enhance the ACE pathway and to provide means for further researching the nature of the energies being captured, stored and transferred.

### BRIEF DESCRIPTION OF THE DRAWINGS

Not Applicable and none included

### DETAILED DESCRIPTION OF THE INVENTION

Microscopic particles present in freshly mined and powdered humic acids suspended in water can display the same type of enhanced kinetic activity as seen when neutral red particles are suspended in ethanol. The kinetic activity of the microscopic particles greatly diminishes over several days and thereafter is no longer discernable. I was particularly interested in observing these movements since comparable movements of particles can be seen in long term tissue culture medium used for the culturing of stealth adapted viruses. Limited restoration of movements of humic acid particles can be achieved by exposure of a humic acid solution to sunlight, but the effects are minimal compared to those seen with initially prepared humic acids solution. I, therefore, investigated whether placing a UV illuminated, activated ethanol plus neutral red solution, in the vicinity dried powdered humic acids would modify the kinetic activity of the powder when subsequently added to water. In a particular embodiment, capsules of humic acids obtained from Morningstar Minerals, Farmington, N. Mex., were placed beneath a plastic bag containing 50 ml of ethanol plus approximately 1 mg of neutral red dye and illuminated with a 13 watt Halco UV-A spiral light bulb. At varying times from 1-12 hours, very small amounts of the humic acids material were added to water and the floating material observed for kinetic movements. Moreover, the dissolving patterns and movements of neutral red dye added to the water containing the humic acids were observed. Humic acids from control capsules not exposed to UV illuminated ethanol and neutral red dye was similarly added to water, with the subsequent addition of neutral red dye. A very clear distinction was seen between the rapid movements of humic acids after being exposed to the UV illuminated ethanol plus neutral red dye compared to the stationary particles seen with humic acids from the control capsules. While enhanced motility of the energy exposed humic acids was less evident when added to regular ethanol, the added humic acids clearly enhanced the kinetic activity of subsequently added neutral red dye. Confirmation was seen with other powdered humic acid preparations and with dilution solutions of humic acids. No effects were seen using capsules with some other powdered products, such as Milk Thistle or Saw Palmetto (dietary supplements obtained from Trader Joe, Monrovia Calif.).

Enhanced NR-Kinetic activity of water and ethanol could, however, be achieved using several preparations of zeolites by prior exposure to UV illuminated ethanol plus neutral red dye. Zeolites are structurally comparable to humic acids as mineral-rich clathrates, but differing in that the host or cage structure of zeolites comprises silica, while that of humic acids comprises carbon. Much of the zeolites used in the present study was obtained from Natural Extracts Australia Pty. Ltd., Hornsby, N.S.W., Australia.

While magnesium oxide powder is soluble in water, beads produced by heating magnesium oxide beyond 900 degrees centigrade are insoluble. There are internet reports of magnesium oxide prills (referring to beads) having beneficial effects on water, but there has been no suggestion of the use of such prills in fluids other than water. I, therefore, determined if magnesium oxide beads (Sigma Aldrich, catalogue 220361, 30 mesh) may be able to directly enhance the NR-Kinetic activity of ethanol and whether this property can be further enhanced by exposure of the beads to a UV illuminated ethanol plus neutral red dye solution. As with humic acids and zeolites, the NR-Kinetic assay showed a marked enhancement of the activity magnesium oxide beads using the UV illuminated ethanol plus neutral red dye solution. It was noted, however, that even unprocessed magnesium oxide beads have quite significant NR-Kinetic assay enhancement activity. The effects were seen using a range of quantities from 1-10 grams of beads per 100 ml of solution and for varying times from 1-24 hours.

The magnesium oxide beads were also used to test whether actual physical contact of the beads with the water or the ethanol was required to observe changes in the NR-Kinetic assay. Simply floating the beads in a plastic container placed onto the surface of water or ethanol was sufficient to achieve an overnight effect. In performing these and other experiments, I needed to be aware of the potential radiating effects of energized solutions on other nearby solutions. Similarly, it was not always possible to obtain water samples, which did not show significant NR-kinetic assay activity. Clearly there are environmental influences acting at different times, even on regular tap water. Still, with patience and detailed observations clear differential can be seen between test and control samples.

Although not as well characterized as with the humic acids, zeolites and magnesium oxide beads, stripped wafers of mica, particles of tourmaline and preparations of diatomaceous earth had both intrinsic ethanol activity (as assessed by the NR-Kinetic assay) and enhanced activity after exposure to UV illuminated ethanol plus neutral red dye solution.

A similar series of studies was performed using humic acids, zeolites and magnesium oxide beads added to alcoholic beverages, rather than absolute ethanol. This was done to help minimize the cost and also to have a consumer product for use of the technology. Among the alcoholic beverages tested were Everclear grain alcohol (151 proof as allowed in California) and Stroh 80 rum (160 proof). Effects essentially comparable to those seen with absolute ethanol were obtained with these high alcohol content beverages. Although, certainly not for human consumption, “denatured alcohol” (cleaning ethanol with added methanol), can also be activated using the described procedures. E85 ethanol (85% ethanol plus gasoline) can also be used but not in the presence of any plastics, which is soluble in gasoline.

The electrolysis of water generates hydrogen gas, oxygen gas and a form of water, which I have termed Water gas. The combination of gases is more commonly termed Brown's gas. I have regularly used a Brown's gas generator available through the internet from a company called Water-to-Gas. A pint sized jar with two electrodes extending from and through the plastic lid, is filled with approximately 300 ml of tap water to which approximately 30 grams of sodium bicarbonate is added. Leads from a 12-volt transformer running at 4.5 amps are attached to the electrodes. Gases evolving from the electrodes (hydrogen, oxygen and Water gas) are captured in a flowing stream of air from a small aquarium pump, which passes along a T shaped tubing connection with its stem passing through the cap. The output tube is typically used to allow the air and electrolysis generated gasses to pass into a liquid, such as ethanol. Rather than bubbling the gas through ethanol, I decided to pass the Water gas through powdered humic acids obtained by emptying a Moringstar Minerals, 800 mg capsules into the gas line. After 30 minutes, small portions of the humic acid powder were added to water and to ethanol. Markedly more movements and inter-particle attractions were shown by the Water gas exposed humic acids, than shown by similar quantities of humic acids not exposed to Water gas. Moreover, enhanced activity of neutral red dye, as assessed in the NR-Kinetic assay, occurred in the water and alcohol solutions using the humic acids exposed to Water gas than with the same type of fluid using non Water gas exposed humic acids. Similar observations were made when zeolites were exposed to exposed to Water gas and directly compared with the activity of zeolites prior to exposure to Water gas. Although, no movements of the Water gas exposed magnesium oxide beads occurred, these beads were clearly able to induce more neutral red activity in both water and ethanol than could non-Water gas exposed magnesium oxide beads. Overall, the bubbling of Water gas through the mineral products was less convenient than placing the materials in close proximity to UV illuminated activated ethanol with added small amounts of neutral red dye.

The relatively (10 minutes) brief UV illumination of sealed plastic containers of activated ethanol plus neutral red dye in a sealed plastic bag placed into a glass of wine was repeatedly been shown to improve the mellowness and taste of the wine. The taste of spirits, but not beer has also been improved in these incidental 6tystudies.

The more important focus has been on understanding the nature of the energy changes occurring in the ethanol and the role of neutral red in the transmission of a distantly acting energizing effect. In addition to enhanced directional solubility of much of the neutral red dye particles and rapidly increased movements of remaining un-dissolved neutral red particles, there is much more intense orange colored UV fluorescence of the activated ethanol solutions. Even the highly energized water will now typically fluoresce and not due to an elevated pH (which can cause fluorescence in neutral red solutions). The amount of neutral red dye required to evoke the fluorescence in activated ethanol is becoming minimal (<0.1 mg/ml). With more neutral red dye, I can commonly observe the formation of many fine strands of neutral red dye in the activated ethanol. Moreover, as noted earlier, many complex ring patterns were observed as the neutral red became deposited from the evaporating ethanol.

The NR-Kinetic assay is enhanced by heat and the activated solutions appear to heat more readily than control solutions from the microscope light. The UV light appears to be an added source of input energy but is not necessary to observe very dynamic kinetic activity.

The other important issue is the usefulness of the described procedures in preparing either drinkable fluids for consumption or activated solutions to be used with neutral red dye in a UV illuminated plastic container placed onto the body. When either the neutral red dye or the UV illumination is not used in the external application of the method, no clinical benefit has been reported. Drinking water and other fluids, which have been activated either with a UV illuminated sealed plastic placed into the fluid has clearly provided clinical benefits. Efforts are underway to assess the usefulness of such fluids in footbaths, whole body bathing, skin sprays and cosmetics. An additional way of energizing water, ethanol and other liquids, as assessed by the NR-Kinetic assay, comprises exposure to ozone, as supplied by an Othrea Toothbrush Sanitizer device. It is clear that multiple procedures exist for activating the kinetic activity of fluids. Many can be used in combination to achieve maximum activation. Each method is subject to refinements, such as showing that powdered moring a leaf powder is superior to olive leaf powder in its activating activity and that moring a seed powder can also be used for fluid activation. The finding that magnesium oxide can be used by itself in activation of ethanol and of Everclear drinking grain alcohol is consistent with mineral based components being the energy delivering active components in moring a and ashitaba leaf powders. (The later powder was obtained as Ashitaba Percent and supplied by Hachi Jo Island Corp. Japan.).

The basic premise of activated fluids providing an effective means of activating the ACE pathway in humans, animals and plants is a fundamental advance in biological science. The fluids are believed to be changed in such as manner that they can continually and more efficiently absorb both conventional and unconventional (e.g. etheric or zero pint) energies from the environment. The present patent application shows that intermediary activation and presumably energy collecting activity can also occur with certain solids. The processes can occur naturally as with moring a and other plant materials and with certain mineral containing organic and inorganic substances, such as humates and zeolites, respectively. Even if lost from these latter materials, energy can be restored using UV illumination of activated ethanol with neutral red dye. Simple mineral containing solids, such as magnesium oxide beads can also show intrinsic energy collecting and transmitting activities, which can be further enhanced.

Beyond its demonstration of clinical utility, activated fluids are anticipated to have many beneficial uses in animal husbandry, correction of animal illnesses, agricultural applications in the growth and quality of plants and industrial uses, such as having anti-corrosion effects, making of better cement, improving the performance of E85 gasoline, etc. Indeed, there are many applications of the methods described within the present application, only some of which are included in the following set of claims.

1. A method of enhancing the energy content and energy transferring activity of mineral-containing materials, including but not limited to humic acids, zeolites and magnesium oxide beads, comprising placing the mineral containing material in close proximity to ultraviolet light illuminated ethanol to which a small amount of neutral red dye has been added; for a sufficient duration of time, such that when the mineral-containing material is itself later added to or placed in close proximity to water or other fluids, the exposed fluids will become more energetically active, as can be shown by more vigorous (kinetic) movements of particles of neutral red dye sprinkled onto a sample of the fluid, when compared to the movement of a similar amount of neutral red dye particles sprinkled onto samples of untreated fluids or fluids exposed to the mineral-containing material, which were not previously exposed to an ultraviolet light illuminated ethanol with neutral red dye solution.

2. A method of enhancing the energy content and energy transferring activity of mineral-containing materials, including but not limited to humic acids, zeolites and magnesium oxide beads, comprising exposing the mineral containing material to gases generated by the electrolysis of water, including what is known as Brown's gas or Water gas, for a sufficient duration of time, such that when the mineral-containing material is itself later added to or placed in close proximity to water or other fluids, the exposed fluids will become more energetically active, as can be shown by more vigorous (kinetic) movements of particles of neutral red dye sprinkled onto a sample of the fluid, when compared to the movement of a similar amount of neutral red dye particles sprinkled onto samples of untreated fluids or fluids exposed to the mineral-containing material, which were not previously exposed to gases generated by the electrolysis of water.

3. A method of energizing a fluid by using an alcohol, such as ethanol, and comprising the addition of neutral red dye to the alcohol, which is then placed in a closed ultraviolet (UV) light transparent container into the fluid to be energized and the container illuminated with UV light for a sufficient duration of time for the fluid to be energized as can be shown by the more vigorous (kinetic) movements of particles of neutral red dye when sprinkled onto a sample of the fluid when compared to the movements of a similar amount of neutral red dye particles sprinkled onto a comparable sample of the untreated fluid.

4. A method of energizing the alternative cellular energy (ACE) pathway in a human or animal subject deficient in this pathway, using ultraviolet (UV) light illumination of an alcohol, such as ethanol with added neutral red dye, and comprising the placement of the alcohol with added neutral red dye into a UV light transparent container, which is placed onto an area of the skin, or into the open mouth of the subject and illuminated with UV light, in such a manner that other areas of the body will begin to show fluorescence or will show an increase in the intensity of any preexisting fluorescence when directly illuminated with UV light during and/or shortly after the procedure.

5. The method of claim 3, for producing an energized fluid, for consumption by, or application to, humans, animals or plants, for the purposes of increasing the levels of alternative cellular energy (ACE) available to the human, animal or plant, beyond that obtainable using comparable amounts of fluids, tested prior to being energized by the method of claim 3.

6. The method of claim 1 in which the energized mineral-containing material is directly used to transfer energy to fluids, for consumption by, application to, or already present within humans, animals of plants and comprising placing the energized mineral-containing material into or in close proximity to the fluid to be energized; including water and alcoholic beverages for consumption; water and other fluids for skin application; and circulating fluids within living humans, animals and plants.

7. The method of claim 1 in which the energized mineral-containing material is directly used to transfer energy to fluids, which are not necessarily suitable for consumption and or direct application to living entities, such as ethanol, which also contains methanol and referred to as denatured alcohol.

8. The method of claim 1 in which the mineral-containing material comprises touramaline, mica or diatomaceous earth.

9. The method of claim 1 in which the mineral-containing material comprises components of plants.

10. The method of claim 2 in which the mineral-containing material comprises touramaline, mica or diatomaceous earth.

11. The method of claim 2 in which the mineral-containing material comprises components of plants.
